(19) United States
(12) Patent Application Publication
(10) Pub. No.: US 2026/0055091 A1
(43) Pub. Date: Feb. 26, 2026

(54) NAPHTHOQUINONE DERIVATIVE AND MEDICAL USE THEREOF

(71) Applicants: KAOHSIUNG MEDICAL UNIVERSITY, Kaohsiung City (TW); CHANG GUNG UNIVERSITY, Taoyuan City (TW); SOOCHOW UNIVERSITY, Taipei City (TW)

(72) Inventors: Chih-Hua Tseng, Kaohsiung City (TW); Chien-Lin Chen, Kaohsiung City (TW); Chia-Yu Fang, Taoyuan City (TW); Shih-Chun Yang, Taipei City (TW)

(73) Assignees: KAOHSIUNG MEDICAL UNIVERSITY, Kaohsiung City, TW (TW); CHANG GUNG UNIVERSITY, Taoyuan City, TW (TW); SOOCHOW UNIVERSITY, Taipei City, TW (TW)

(21) Appl. No.: 18/809,353

(22) Filed: Aug. 20, 2024

(51) Int. Cl. C07D 413/06; A61K 31/423; A61P 31/04; A61P 31/10

(52) U.S. Cl. CPC C07D 413/06; A61K 31/423; A61P 31/04; A61P 31/10

(57) ABSTRACT

Naphthoquinone derivatives have anti-inflammatory, antifungal and antibacterial effects. The naphthoquinone derivatives are able to effectively inhibit expression of inflammatory factors and be used for treating infections caused by Candida and Staphylococcus. In addition, an anti-inflammatory method is provided which includes administering the naphthoquinone derivatives for treatment, and a method of treating microbial infection includes administering the naphthoquinone derivatives for treatment.

### FIELD OF THE INVENTION

The present invention relates to a naphthoquinone derivative, which has anti-inflammatory, antifungal, and antibacterial effects.

### DESCRIPTION OF PRIOR ARTS

Fungal infections affect approximately 2 million people worldwide each year and are particularly common in patients with low immunity. For example, patients undergoing organ transplantation, cancer chemotherapy, and patients in intensive care units are all susceptible to fungal infections. Among all pathogens, Candida is one of the most common pathogens, causing skin, muscle, and systemic infections. When fungal infections are not treated in time, the mortality rate is nearly 60%. Candida genus can be subdivided into different strains, and Candida albicans is the most common one. According to the information provided by the Taiwan Ministry of Health and Welfare, between 2013 and 2022, infections caused by Candida albicans were the top two infections in intensive care units, and drug resistance has been developed to some of the antifungal drugs that are currently available clinically. Therefore, there is an urgent need to develop new antifungal drugs.

Naphthoquinone is a natural compound that exists in a variety of plants and has also been found to have many biological activities, such as anti-cancer, antibacterial and anti-inflammatory activities. Therefore, naphthoquinone derivatives may have the potential of being antibacterial or anti-inflammatory.

### SUMMARY OF THE INVENTION

The present invention provides a naphthoquinone derivative, which has anti-inflammatory, antifungal, and antibacterial effects.

The present invention provides an anti-inflammatory method, which comprises administering a naphthoquinone derivative for treatment.

The present invention further provides a method for treating microbial infections, which comprises administering a naphthoquinone derivative for treatment.

### BRIEF DESCRIPTION OF THE DRAWINGS

FIG. 1 shows a preparation process of naphtho[2,1-d]oxazol-5-ol derivatives. ACN: acetonitrile. KI: potassium iodide. DMF: Dimethylformamide.

FIG. 2 shows a preparation process of naphtho[2,1-d]oxazole-4,5-dione derivatives. DCE: Dichloroethane.

FIG. 3 shows the cytotoxicity of the naphtho[2,1-d]oxazol-5-ol derivatives tested on a CCK8 platform at a concentration of 10 μM. CTL: Control group.

FIG. 4 shows the effect of the naphtho[2,1-d]oxazol-5-ol derivatives on inhibiting the expression of the inflammatory factor IL-6. CTL: Control group.

FIG. 5 shows the effect of naphtho[2,1-d]oxazol-5-ol derivatives on inhibiting the expression of the inflammatory factor IL-8. CTL: Control group.

FIG. 6 shows the effect of naphtho[2,1-d]oxazol-5-ol derivatives on inhibiting the expression of the chemokine CXCL-1. CTL: Control group.

FIG. 7 shows the treatment condition of naphtho[2,1-d]oxazole-4,5-dione derivatives in mice infected with MRSA. CTL: Control group. PBS: Phosphate buffered saline.

### DETAILED DESCRIPTION OF THE INVENTION

The present invention adopts the principle of bioequivalent isomers to design and synthesize naphtho[2,1-d]oxazol-5-ol and naphtho[2,1-d]oxazole-4,5-dione derivatives. Further, to evaluate their biological activities, a 1,2,3-triazole ring is introduced through click chemistry.

The results of anti-inflammation experiments show that naphtho[2,1-d]oxazol-5-ol has anti-inflammatory activity. The cytotoxicity of these compounds is tested through a CCK8 platform, and it is found that most of the compounds have a cell survival rate of more than 80% at a concentration of 10 μM. In addition, they have inhibitory effects on the expression of the cytokines IL-6, IL-8, and chemokine CXCL-1. Among them, Compounds 18a and 18e show the best anti-inflammatory effects, and their inhibitory abilities against three cytokines are over 80% at a concentration of 10 μM.

On the other hand, naphtho[2,1-d]oxazole-4,5-dione has anti-Candida albicans and anti-methicillin-resistant Staphylococcus aureus (MRSA) properties. Compared to the antibacterial effects of commercially available fluconazole and oxacillin, the antibacterial activities of all compounds of the present invention are significantly improved. Among them, Compound 24j has a minimum inhibitory concentration (MIC) of 11.6 μM for MRSA and is expected to become a leading compound for anti-MRSA drugs.

As used herein, the terms “a” or “an” are used to describe elements and components of the present invention. This terminology is used only for convenience and to provide a basic concept of the present invention. Furthermore, this description should be understood to include one or at least one, and unless the context clearly dictates otherwise, singular terms should include plural terms, and plural terms should include singular. When used in conjunction with the word “comprising” in the claims, the term “a” or “an” may mean one or more than one.

The term “or” as used herein may mean “and/or”.

The present invention provides a compound or a pharmaceutically acceptable salt thereof, wherein the compound has a structure of formula (I):

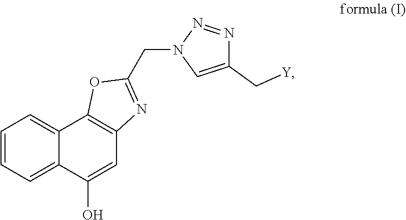

wherein Y is OH, or

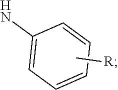

and
R is H, OH, halogen, C₁₋₁₀ alkyl or C₁₋₁₀ alkoxyl.

The compound of the present invention also comprise a pharmaceutically acceptable salt thereof. These salts comprise pharmaceutically acceptable alkali salts thereof, including alkali metal salts (for example, sodium salts, potassium salts), alkaline earth metal salts (for example, calcium salts, magnesium salts), ammonium salts, and salts formed with organic bases (for example, dicyclohexylamine and N-methyl-D-glucosamine).

In one embodiment, the halogen comprises fluorine (F), chlorine (Cl), bromine (Br), iodine (I) and astatine (At). In a preferred embodiment, the halogen is fluorine or chlorine. In a more preferred embodiment, the halogen is fluorine.

As used herein, the term “C₁₋₁₀ alkyl” refers to a substituted or unsubstituted linear or branched saturated hydrocarbon group containing from 1 to 10 carbon atoms. In a preferred embodiment, the C₁₋₁₀ alkyl comprises C₁₋₈ alkyl. More preferably, the alkyl is a substituted or unsubstituted C₁₋₆ alkyl, including but not limited to a substituted or unsubstituted methyl (—CH3 or -Me), ethyl, n-propyl, isopropyl, n-butyl, sec-butyl, tert-butyl, n-pentyl, isopentyl, n-hexyl, etc. In another embodiment, the C₁₋₁₀ alkyl comprises C₁₋₄ alkyl. In a preferred embodiment, the C₁₋₁₀ alkyl comprises methyl.

As used herein, the term “C₁₋₁₀ alkoxy” refers to a group formed by connecting a substituted or unsubstituted C₁₋₁₀ alkyl to an oxygen atom. In a preferred embodiment, the C₁₋₁₀ alkoxy comprises C₁₋₈ alkoxy. More preferably, the alkyl is a substituted or unsubstituted C₁₋₆ alkyl, including but not limited to substituted or unsubstituted methoxy (—OCH3 or —OMe), ethoxy, propoxy, butoxy, pentyloxy, hexyloxy, etc. In another embodiment, the C₁₋₁₀ alkoxy comprises C₁₋₄ alkoxy. In a preferred embodiment, the C₁₋₁₀ alkoxy comprises methoxy.

In another embodiment, the R is H, OH, fluorine or methoxy.

The present invention further provides a use of a compound or a pharmaceutically acceptable salt thereof for preparing anti-inflammatory drugs, wherein the compound has the structure of formula (I).

The present invention further provides an anti-inflammatory method, which comprises administering a composition comprising a compound or a pharmaceutically acceptable salt thereof to a subject suffering from inflammation, wherein the compound has the structure of formula (I).

As used herein, the term “anti-inflammation” refers to a use of a compounds to treat, inhibit or reduce symptoms and occurrences of inflammatory responses. In some aspects, the compound having the structure of formula (I) can inhibit inflammatory factors for anti-inflammation. In one embodiment, the compound having the structure of formula (I) inhibits expression of inflammatory factors, and the inflammatory factors include IL-6, IL-8 and CXCL-1.

As used herein, the term “subject” refers to an animal, including a human. Therefore, the term “subject” comprises any mammals that can benefit from the methods of the present invention. The term “mammal” refers to all members of the class Mammalia. In one embodiment, the subject is a human.

The present invention also provides an anti-inflammatory composition, which comprises the compound having the structure of formula (I) or a pharmaceutically acceptable salt thereof, and a pharmaceutically acceptable carrier, excipient or diluent agent.

Therefore, the present invention comprises administering a therapeutically effective amount of the compound having the structure of formula (I) to the subject suffering from inflammation for treatment. The term “therapeutically effective amount” as used herein refers to an amount of a drug that can alleviate or eliminate a disease or symptoms in a subject, or can prophylactically inhibit or prevent occurrences of a disease or symptoms. In one embodiment, the therapeutically effective amount of the compound having the structure of formula (I) ranges from 1 to 10 μM.

In addition, the present invention provides a compound or a pharmaceutically acceptable salt thereof, wherein the compound has a structure of formula (II):

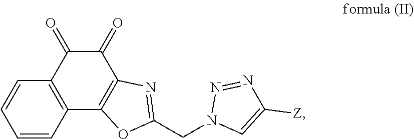

wherein Z is C₁₋₁₀ alkyl, C₁₋₃ alkyl-OH, or

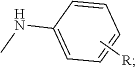

and
R is H, OH, halogen, C₁₋₁₀ alkyl or C₁₋₁₀ alkoxy.

In one embodiment, the Z is C₃₋₁₀ alkyl. In a preferred embodiment, the Z is C₃₋₁₀ alkyl. In a more preferred embodiment, the Z is C₃₋₅ alkyl.

In another embodiment, the Z is C₁ alkyl-OH.

In one embodiment, the R is H, fluorine, chlorine, methyl or methoxy.

The present invention further provides a use of a compound or a pharmaceutically acceptable salt thereof for preparing a drug for treating microbial infections, wherein the compound has the structure of formula (II), and the microorganism comprises Candida or Staphylococcus.

The present invention further provides a method for treating microbial infections, which comprises administering a composition comprising a compound or a pharmaceutically acceptable salt thereof to a subject suffering from microbial infections, wherein the compound has the structure of formula (II), and the microorganism comprises Candida or Staphylococcus.

The term “treating” is meant to include alleviation or elimination of a disorder, disease, or one or more symptoms associated with the disorder, disease, or conditions; or alleviation or elimination of the cause of the disorder, disease, or symptoms.

In the present invention, the microbial infection is selected from bacterial (for example, Staphylococcus aureus) or fungal (for example, Candida) infections, preferably drug-resistant bacterial infections or drug-resistant fungal infections.

In some aspects, the fungal infections can be caused or exacerbated by Candida. In one embodiment, the Candida includes drug-resistant and non-drug-resistant C. albicans, C. parapsilosis, C. tropicalis, C. glabrata, C. guilliermondii, or C. krusei. In a preferred embodiment, the Candida albicans is drug-resistant Candida albicans.

In some aspects, the bacterial infections can be caused or exacerbated by Staphylococcus. In one embodiment, the staphylococci include S. aureus, S. haemolyticus, S. hominis, S. intermedius, and S. saprophyticus, or S. saccharolyticus. In another embodiment, the staphylococcus is drug-resistant staphylococcus. In one preferred embodiment, the drug-resistant Staphylococcus is resistant to methicillin, vancomycin, glycopeptide antibiotics, penicillin or daptomycin. In one embodiment, the Staphylococcus aureus is drug-resistant Staphylococcus aureus. In one preferred embodiment, the Staphylococcus aureus comprises methicillin-resistant Staphylococcus aureus (MRSA), glycopeptide intermediate susceptible Staphylococcus aureus (GISA), Vancomycin-resistant Staphylococcus aureus (VRSA), or vancomycin intermediate-resistant Staphylococcus aureus (VISA).

Therefore, the present invention comprises administering a therapeutically effective amount of the compound having the structure of formula (II) to the subject suffering from microbial infections for treatment. In one embodiment, the therapeutically effective amount of the compound having the structure of formula (II) ranges from 10 to 200 μM.

In the present invention, the compound having the structure of formula (II) can inhibit the activity of Candida albicans and methicillin-resistant Staphylococcus aureus. When the R on the compound having the structure of formula (II) is H or fluorine, the compound has a better antibacterial effect against Candida albicans. When the R on the compound having the structure of formula (II) is fluorine, chlorine, methyl or methoxy, the compound has a better antibacterial effect against methicillin-resistant Staphylococcus aureus. Therefore, the compound having the structure of formula (II) can be used clinically as an antifungal agent and an antibacterial agent.

The present invention also provides an antimicrobial infection composition, which comprises the compound having the structure of formula (II) or a pharmaceutically acceptable salt thereof and a pharmaceutically acceptable carrier, excipient or diluent, wherein the microorganisms include Candida or Staphylococcus.

As used herein, “pharmaceutically acceptable carrier, excipient or diluent” refers to a carrier and excipient or diluent that is made by a pharmaceutically acceptable material or substrates, such as a liquid or solid filler, stabilizer, dispersant, suspensions, thickener, solvent or encapsulating material, that acts to transport the active ingredients of the present invention and makes the active ingredients play their function in a patient's body. Each carrier must be compatible with various formulation ingredients of the composition (including the compound of formula (I) or formula (II) of the present invention) so as not to adversely affect the patient.

Accordingly, the naphtho[2,1-d]oxazole-5-ol and naphtho[2,1-d]oxazole-4,5-dione derivatives developed by the present invention have anti-inflammatory and anti-pathogenic bacterial (for example, Candida and Staphylococcus) effects.

### Examples

The following examples are non-limiting and merely represent aspects and features of the present invention.

The present invention adopted the principle of bioequivalent isomers to design and synthesize naphtho[2,1-d]oxazol-5-ol and naphtho[2,1-d]oxazole-4,5-dione derivatives. Further, to evaluate their biological activities, a 1,2,3-triazole ring was introduced through click chemistry.

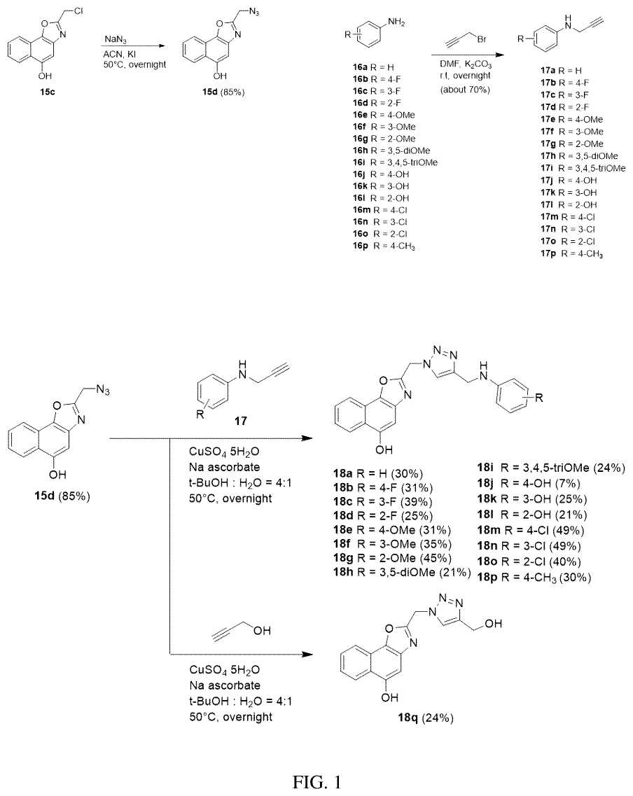

FIG. 1 is a preparation process of the naphtho[2,1-d]oxazole-5-ol derivatives. The preparation process of Compound 18e is provided by the present invention as exemplar description.

### Preparation of Compound 18e

Compound 15d (0.240 g, 1.0 mmol) and the synthesized 4-methoxy-N-(prop-2-yn-1-yl)aniline) (0.241 g, 1.5 mmol) were dissolved in 8 ml of tert-butanol and stirred for 10 minutes. Sodium ascorbate (0.100 g, 0.50 mmol) and copper sulfate pentahydrate (0.130 g, 0.50 mmol) were dissolved in 1 ml of water, respectively, and then added to the tert-butanol solution prepared above, stirred at room temperature for 12 hours (monitored with the thin layer chromatography (TLC)). Upon completion of the rection, a vacuum concentrator was used to dry tert-butanol, and then dichloromethane, methanol (DCM: MeOH=4:1, 50 ml×3) and water were used for extraction. The organic layer was collected, water was removed with anhydrous magnesium sulfate, and then an earthy yellow solid Compound 18e (0.124 g, yield 31%) was obtained by using silica gel column chromatography (using methylene chloride: methanol=50:1 as an eluent) for separation.

¹H NMR (400 MHz, DMSO-d₆) δ 10.41 (s, 1H, OH), 8.27 (d, J=8 Hz, 1H, Ar—H), 8.17 (s, 1H, H-triazole), 8.03 (d, J=8 Hz, 1H, Ar—H), 7.71-7.67 (m, 1H, Ar—H), 7.59-7.55 (m, 1H, Ar—H), 7.10. (s, 1H, Ar—H), 6.75-6.70 (m, 2H, Ar—H), 6.63-6.61 (m, 2H, Ar—H), 6.08 (s, 2H, CH₂), 5.68 (s, 1H, NH), 4.29 (s, 2H, CH₂), 3.62 (s, 3H, OCH₃)

¹³C NMR (400 MHz, DMSO-d₆) δ 159.50, 151.36, 150.96, 146.51, 142.50, 139.71, 137.14, 127.79 (2C), 125.02, 123.81, 123.55, 123.30, 119.70, 119.44, 114.51 (2C), 113.52 (2C), 99.01, 55.24, 46.51

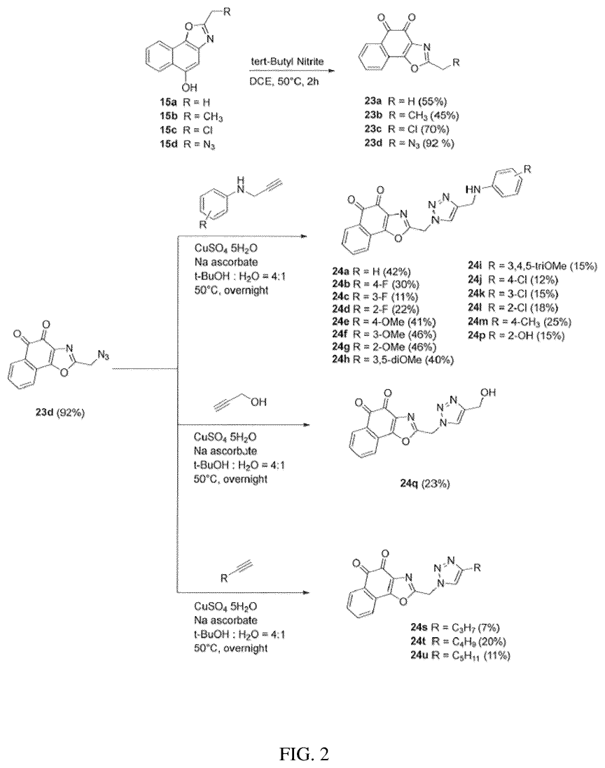

FIG. 2 is a preparation process of the naphtho[2,1-d]oxazole-4,5-dione derivatives. The preparation processes of Compounds 24j and 24m are provided by the present invention as exemplary description.

### Preparation of Compound 24j

Compound 23d (0.254 g, 1.0 mmol) and the synthesized 4-chloro-N-(prop-2-yn-1-yl)aniline (0.248 g, 1.5 mmol) were dissolved in 8 ml of the tert-butanol and stirred for 10 minutes. Sodium ascorbate (0.100 g, 0.50 mmol) and copper sulfate pentahydrate (0.130 g, 0.50 mmol) were dissolved in 1 ml of water, respectively, and then added to the tert-butanol solution prepared above, stirred at room temperature for 12 hours (monitored with TLC). Upon completion of the reaction, a vacuum concentrator was used to dry the tert-butanol, and then dichloromethane and methanol (DCM: MeOH=4:1, 50 ml×3) and water were used for extraction. The organic layer was collected, water was removed with anhydrous magnesium sulfate, and then an earthy yellow solid Compound 24j (0.051 g, yield 12%) was obtained by using silica gel column chromatography (using methylene chloride: methanol=50:1 as the eluent) for separation.

¹H NMR (400 MHz, DMSO-d₆) δ 8.18 (s, 1H, H-triazole), 8.01 (d, J=8 Hz, 1H, Ar—H), 7.80-7.75 (m, 1H, Ar—H), 7.65-7.61 (m, 2H, Ar—H), 7.10-7.07 (m, 2H, Ar—H), 6.67-6.64 (m, 2H, Ar—H), 6.37-6.36 (m, 1H, NH), 6.05 (s, 2H, CH₂), 4.34 (d, J=4 Hz, 2H, CH₂)

¹³C NMR (400 MHz, DMSO-d₆) δ 178.43, 172.13, 159.28, 157.77, 147.20, 145.95, 134.82 (2C), 133.87, 131.09, 130.31, 129.56, 128.53 (2C), 124.98, 123.95, 122.48, 119.31, 113.72 (2C), 45.85

### Preparation of Compound 24m

Compound 23d (0.254 g, 1.0 mmol) and the synthesized 4-methyl-N-(prop-2-yn-1-yl)aniline (0.217 g, 1.5 mmol) were dissolved in 8 ml of the tert-butanol and stirred for 10 minutes. Sodium ascorbate (0.100 g, 0.50 mmol) and copper sulfate pentahydrate (0.130 g, 0.50 mmol) were dissolved in 1 ml of water, respectively, and then added to the tert-butanol solution prepared above, stirred at room temperature for 12 hours (monitored with TLC). Upon completion of the reaction, a vacuum concentrator was used to dry the tert-butanol, and then dichloromethane and methanol (DCM: MeOH=4:1, 50 ml×3) and water were used for extraction. The organic layer was collected, water was removed with anhydrous magnesium sulfate, and then an earthy yellow solid Compound 24m (0.099 g, yield 25%) was obtained by using silica gel column chromatography (using methylene chloride: methanol=50:1 as the eluent) for separation.

¹H NMR (400 MHz, DMSO-d₆) δ 8.24 (s, 1H, H-triazole), 8.01 (d, J=8 Hz, 1H, Ar—H), 7.80-7.76 (m, 2H, Ar—H), 7.68-7.61 (m, 3H, Ar—H), 7.35-7.31 (m, 3H, Ar—H, NH), 6.04 (s, 2H, CH₂), 5.33 (s, 2H, CH₂), 2.34-2.33 (m, 3H, CH₃)

¹³C NMR (400 MHz, DMSO-d₆) δ 178.43, 172.11, 159.10, 157.81, 140.74, 138.71, 137.17, 134.85 (2C), 133.86, 131.11, 130.30, 129.94 (2C), 129.58, 125.28 (2C), 122.51, 120.22 (2C), 45.93, 20.50

### 1. Safety Test of Naphtho[2,1-d]Oxazole-5-Ol Derivatives

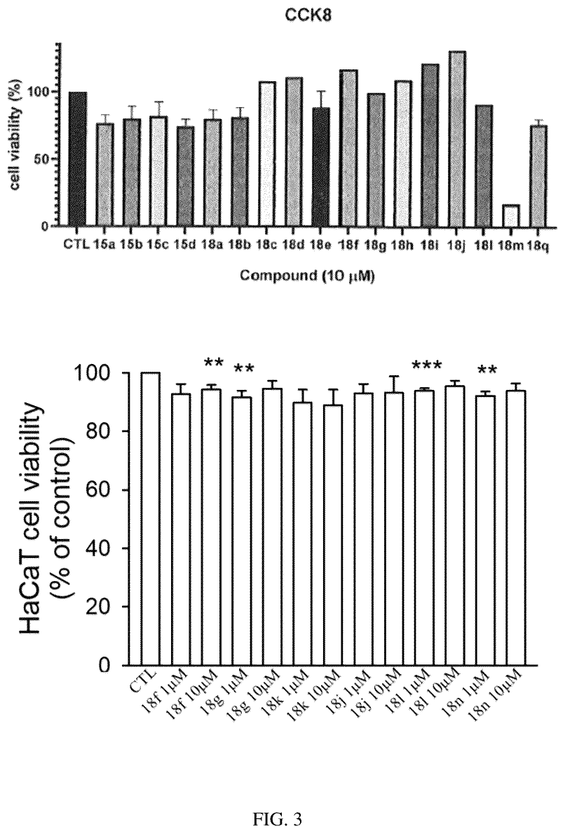

Before testing whether the compounds could inhibit the expression of cytokines, the present invention first conducted safety tests of the compounds. The present invention used the CCK8 platform to evaluate the cytotoxicity of the compounds. As shown in FIG. 3, Compound 18m was the most toxic compound with only about 15% cell survival rate. The rest of the compounds had cell survival rate close to 80% at a concentration of 10 μM. Therefore, the present invention chose to test the inhibitory effect of the compounds on cytokine expression at the concentration of 10 μM.

### 2. Inhibition of the Expression of IL-6, IL8 and CXCL-1 by Naphtho[2,1-d]Oxazole-5-Ol Derivatives

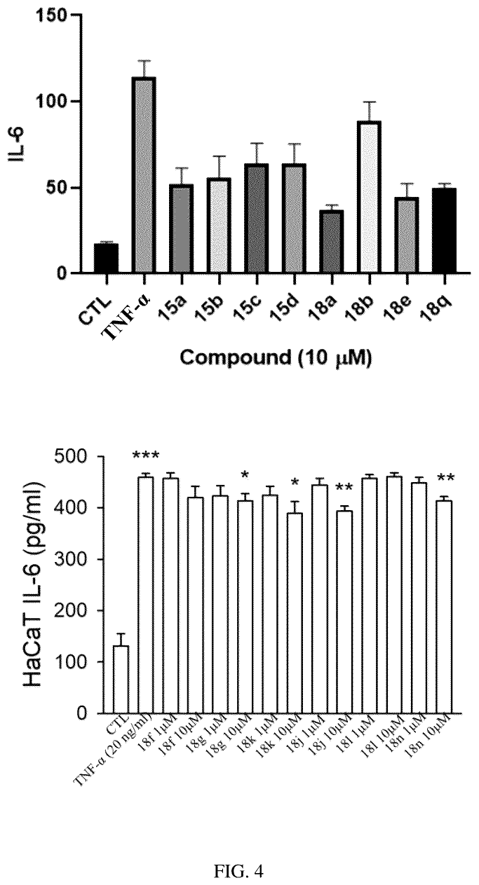

FIG. 4 shows the inhibitory effect of naphtho[2,1-d]oxazole-5-ol derivatives on the expression of IL-6. Eight compounds were tested (15a, 15b, 15c, 15d, 18a, 18b, 18e, 18q), and all showed inhibitory effects on the expression of IL-6 caused by tumor necrosis factor (TNF-α), among which Compounds 18a and 18e most effectively decreased the expression of IL-6 in inflammatory cells. In addition, Compounds 18f, 18g, 18k, 18j, 181, and 18n were able to inhibit the expression of IL-6 at a concentration from 1 μM to 10 μM, among which Compounds 18k and 18j had the best effects at 10 μM.

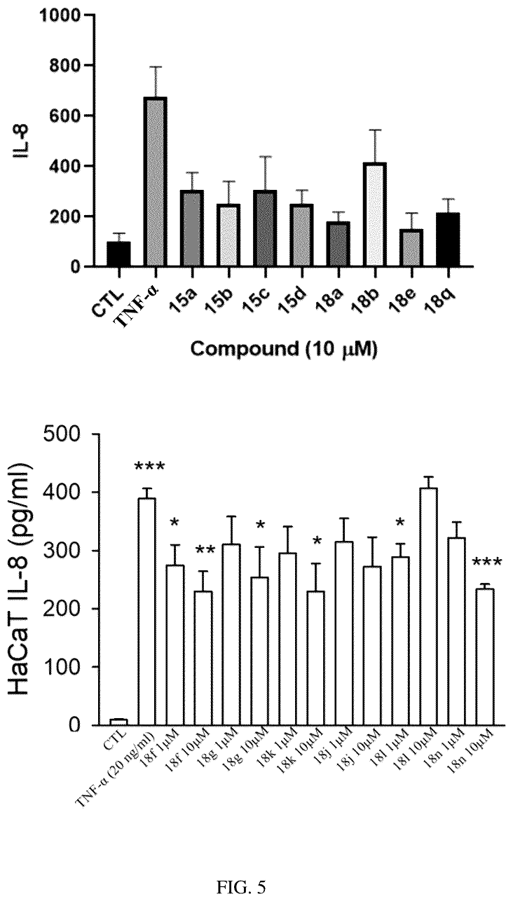

The inhibitory effect on IL-8 is shown in FIG. 5. Compared to IL-6, eight compounds had relatively better inhibitory activities against IL-8. Similarly, Compounds 18a and 18e also performed the best among all eight tested compounds. Compounds 18f, 18g, 18k, 18j, 181, and 18n also showed inhibitory effects at a concentration from 1 μM to 10 μM, and Compound 18n was the most effective one.

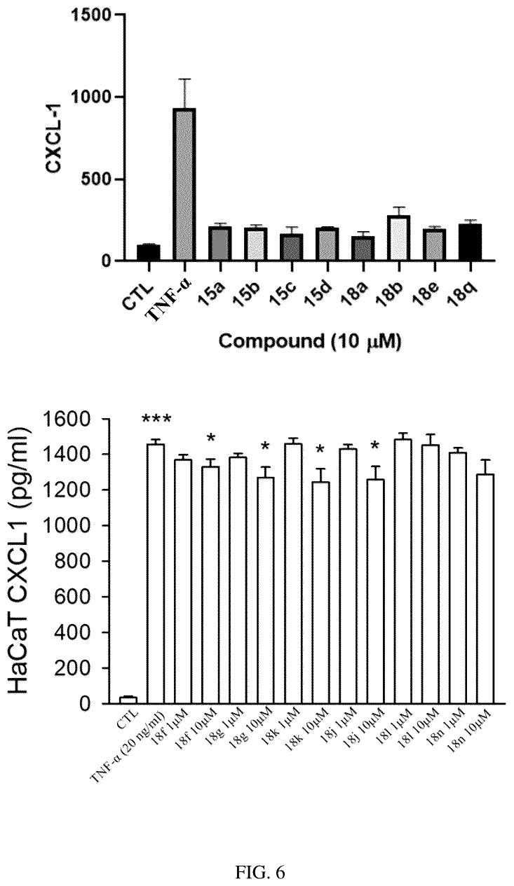

CXCL-1 is a common chemokine in cells. In inflammatory cells, the expression level of CXCL-1 also tends to increase. According to the activity test results shown in FIG. 6, all eight tested compounds had good inhibitory effects on CXCL-1. At a concentration of 10 μM, Compounds 18a and 18e were able to inhibit the expression of CXCL-1 to the greatest extent. In addition, Compounds 18f, 18g, 18j, and 18n also had good anti-inflammatory effects at a concentration of 10 μM.

3. Inhibitory Activity of Naphtho[2,1-d]Oxazole-4,5-Dione Derivatives on Drug-Resistant Candida albicans

The inhibition zone test was used to evaluate the synthesized compounds' anti-Candida albicans activities. The inhibition zone results of the synthesized compounds against two drug-resistant Candida albicans strains, ATCC 90029 and ATCC 10231, are shown in Tables 1 to 6, and all values represent an average of three experiments.

As shown in Tables 1 and 2, the preliminary screening results showed that Compounds 23a-23d all had relatively good antibacterial abilities. In particular, Compounds 23a and 23b, at a concentration of 5000 μg/ml, had the inhibition zone sizes of nearly 20 mm or more for both strains.

TABLE 1
The inhibition zone size (mm) of Compounds 23a-23d against ATCC
90029

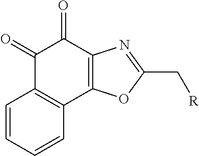

ATCC 90029

| | | 5000 | 2500 | 1250 | 625 | 312 | 125 |
|---|---|---|---|---|---|---|---|
| Compound | R | μg/ml | μg/ml | μg/ml | μg/ml | μg/ml | μg/ml |
| 23a | H | 24.66 | 23.33 | 20.83 | 18.17 | 16.83 | 14.33 |
| 23b | CH₃ | 25.17 | 21.83 | 19.00 | 17.67 | 15.83 | 12.50 |
| 23c | Cl | 19.67 | 18.67 | 16.00 | 13.33 | 11.67 | 10.17 |
| 23d | N₃ | 19.33 | 18.17 | 15.50 | 14.83 | 12.00 | 10.33 |

TABLE 2
The inhibition zone sizes (mm) of Compounds 23a-23d against ATCC
10231

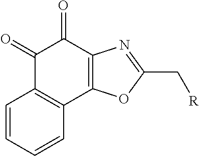

ATCC 10231

| | | 5000 | 2500 | 1250 | 625 | 312 | 125 |
|---|---|---|---|---|---|---|---|
| Compound | R | μg/ml | μg/ml | μg/ml | μg/ml | μg/ml | μg/ml |
| 23a | H | 19.67 | 15.33 | 9.33 | 5.67 | 5.33 | 7.00 |
| 23b | CH₃ | 24.33 | 17.00 | 10.67 | 6.00 | 5.00 | 4.67 |
| 23c | Cl | 10.00 | 7.00 | 5.00 | 5.00 | 5.33 | 5.00 |
| 23d | N₃ | 6.33 | 6.00 | 5.00 | 5.00 | 5.00 | 8.00 |

The present invention further modified Compound 23d by connecting 1,2,3-triazole to obtain a series of Compound 24 derivatives. As shown in Tables 3 and 4, the Compound 24 series showed relatively good inhibition zone ability against two strains of Candida albicans, and the inhibitory ability against ATCC90029 was better than that against ATCC10231.

TABLE 3
The inhibition zone sizes (mm) of Compounds 24a-24q against ATCC
90029

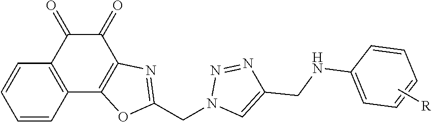

ATCC 90029

| | | 5000 | 2500 | 1250 | 625 | 312 | 125 |
|---|---|---|---|---|---|---|---|
| Compound | R | μg/ml | μg/ml | μg/ml | μg/ml | μg/ml | μg/ml |
| 24a | H | 13.33 | 12.83 | 11.83 | 10.33 | 9.83 | 8.00 |
| 24b | 4-F | 13.50 | 13.33 | 12.33 | 10.00 | 8.83 | 7.50 |
| 24c | 3-F | 12.50 | 11.67 | 10.67 | 7.83 | 9.75 | 8.33 |
| 24d | 2-F | 11.00 | 8.33 | 8.00 | 11.00 | 7.00 | 6.00 |
| 24e | 4-OMe | 11.67 | 11.00 | 10.33 | 9.33 | 9.00 | 7.33 |
| 24f | 3-OMe | 11.00 | 9.75 | 8.17 | 8.67 | 8.67 | 8.50 |
| 24g | 2-OMe | 9.50 | 9.17 | 8.50 | 8.67 | 8.17 | 8.17 |
| 24h | 3,5- | 10.67 | 10.08 | 10.67 | 7.67 | 8.83 | 8.17 |
| | diOMe | | | | | | |
| 24i | 3,4,5- | 10.33 | 11.00 | 9.75 | 9.67 | 10.67 | 10.00 |
| | triOMe | | | | | | |
| 24j | 4-C1 | 15.75 | 12.33 | 11.00 | 10.33 | 9.17 | 7.50 |
| 24k | 3-C1 | 11.00 | 7.00 | 8.00 | 10.00 | 10.50 | 0.00 |
| 241 | 2-C1 | 10.00 | 8.50 | 7.50 | 9.5 | 11.00 | 0.00 |
| 24m | 4-Me | 13.00 | 11.83 | 11.33 | 7.83 | 8.17 | 9.17 |
| 24p | 2-OH | 11.67 | 13.00 | 17.00 | 12.00 | 10.00 | 0.00 |
| 24q | | 13.83 | 12.83 | 11.17 | 10.00 | 9.33 | 7.00 |

TABLE 4
The inhibition zone sizes (mm) of Compounds 24a-24q against ATCC
10231

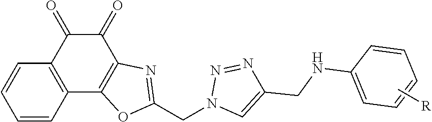

ATCC 10231

| | | 5000 | 2500 | 1250 | 625 | 312 | 125 |
|---|---|---|---|---|---|---|---|
| Compound | R | μg/ml | μg/ml | μg/ml | μg/ml | μg/ml | μg/ml |
| 24a | H | 6.00 | 6.00 | 6.00 | 5.00 | 9.00 | 13.00 |
| 24b | 4-F | 6.00 | 6.00 | 6.00 | 6.00 | 4.43 | 12.00 |
| 24c | 3-F | 9.17 | 9.00 | 10.00 | 7.67 | 8.73 | 9.33 |
| 24d | 2-F | 13.00 | 7.50 | 0.00 | 8.00 | 7.00 | 0.00 |
| 24e | 4-OMe | 6.00 | 5.00 | 5.00 | 5.00 | 4.33 | 4.00 |
| 24f | 3-OMe | 9.58 | 10.67 | 11.08 | 8.00 | 8.33 | 8.83 |
| 24g | 2-OMe | 8.58 | 9.50 | 9.50 | 8.58 | 9.33 | 9.17 |
| 24h | 3,5- | 8.00 | 8.67 | 9.58 | 7.67 | 8.50 | 8.00 |
| | diOMe | | | | | | |
| 24i | 3,4,5- | 8.83 | 8.83 | 10.58 | 7.33 | 7.33 | 7.67 |
| | triOMe | | | | | | |
| 24j | 4-Cl | 9.50 | 9.67 | 10.00 | 8.50 | 9.83 | 9.17 |
| 24k | 3-Cl | 7.00 | 9.00 | 10.33 | 7.33 | 7.67 | 10.33 |
| 241 | 2-Cl | 9.00 | 8.00 | 9.33 | 8.33 | 8.33 | 7.67 |
| 24m | 4-Me | 7.67 | 9.25 | 10.33 | 7.67 | 8.33 | 8.33 |
| 24p | 2-OH | 7.50 | 7.67 | 8.67 | 6.00 | 8.00 | 9.50 |
| 24q | | 6.33 | 5.67 | 5.67 | 4.67 | 5.00 | 9.33 |

As shown in Tables 5 and 6, the inhibition zone sizes of some of the compounds (such as Compound 24s-24u) at high concentrations were smaller than those at low concentrations. The present invention speculated that the solubility of the compounds at high concentrations caused precipitation on algin, thereby affecting the antibacterial abilities.

TABLE 5
The inhibition zone sizes (mm) of Compounds 24s-24u against ATCC
90029

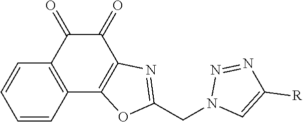

ATCC 90029

| | | 5000 | 2500 | 1250 | 625 | 312 | 125 |
|---|---|---|---|---|---|---|---|
| Compound | R | μg/ml | μg/ml | μg/ml | μg/ml | μg/ml | μg/ml |
| 24s | C₃H₇ | 13.33 | 12.33 | 9.00 | 10.33 | 8.00 | 8.00 |
| 24t | C₄H₉ | 13.00 | 10.00 | 8.50 | 11.67 | 11.50 | 14.00 |
| 24u | C₅H₁₁ | 10.33 | 11.67 | 9.00 | 8.33 | 9.33 | 10.67 |

TABLE 6
The inhibition zone sizes (mm) of Compounds 24s-24u against ATCC
10231

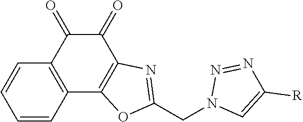

ATCC 10231

| | | 5000 | 2500 | 1250 | 625 | 312 | 125 |
|---|---|---|---|---|---|---|---|
| Compound | R | μg/ml | μg/ml | μg/ml | μg/ml | μg/ml | μg/ml |
| 24s | C₃H₇ | 7.00 | 8.33 | 10.00 | 7.33 | 9.67 | 10.00 |
| 24t | C₄H₉ | 11.00 | 10.00 | 10.50 | 7.67 | 8.33 | 9.67 |
| 24u | C₅H₁₁ | 8.00 | 11.00 | 11.33 | 9.00 | 12.67 | 12.33 |

The above inhibition zone test was only preliminary screening of the antibacterial ability of the compounds. In order to remove the influence of external factors such as solubility and diffusion conditions, the present invention further tested the minimum inhibitory concentration (MIC) and minimum fungicidal concentration (MFC) of the three series of synthesized compounds against two drug-resistant strains.

The MIC and MFC values of the compounds against the drug-resistant Candida albicans strains ATCC 90029 and ATCC 10231 are shown in Table 7 to Table 9, and fluconazole, commonly used in clinical practices today, was used as a control group.

As shown in Table 7, Compounds 23a-23d showed quite good antibacterial abilities. Among them, for ATCC 90029, Compound 23b showed the best antibacterial ability, and the MIC and MFC values were 171.9 and 343.8 μM, respectively. Although its antibacterial ability was not as good as that of fluconazole, the MFC value showed that the fungicidal ability of Compound 23b was increased by 6 times. On the other hand, Compound 23c showed relatively outstanding antibacterial ability against ATCC 10231, and the MIC and MFC values were 210.3 and 473.2 μM, respectively. Compared with fluconazole, the bacteriostatic and fungicidal abilities of Compound 23c were increased by 19 times and 9 times, respectively.

TABLE 7
The MIC and MFC values of Compounds 23a-23d against drug-
resistant Candida albicans

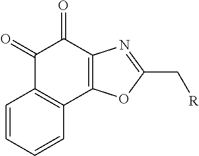

| | | ATCC 90029 | | ATCC 10231 | |
|---|---|---|---|---|---|
| | | MIC | MFC | MIC | MFC |
| Compound | R | (μM) | (μM) | (μM) | (μM) |
| 23a | H | 183.2 | 732.9 | 229.0 | 549.7 |
| 23b | CH₃ | 171.9 | 343.8 | 229.2 | 429.8 |
| 23c | Cl | 315.5 | 578.4 | 210.3 | 473.2 |
| 23d | N₃ | 614.6 | 717.1 | 1229.3 | 1843.9 |

| | | | | |
|---|---|---|---|---|
| Fluconazole | 4.0 | 2040.7 | 4081.4 | 4081.4 |

As shown in Tables 8 and 9, the antibacterial abilities of the Compound 24 series were also significantly improved as compared to fluconazole. For ATCC 90029, Compounds 24a and 24b had relatively good antibacterial effects, the MIC values were 405.6 and 516.6 μM, and the MFC values were 608.2 and 581.0 μM. Compared to fluconazole, the fungicidal abilities of Compounds 24a and 24b were increased by about three times. On the other hand, the Compound 24 series was 2-4 times better than fluconazole in terms of antibacterial and fungicidal abilities against ATCC 10231.

TABLE 8
The MIC and MFC values of Compounds 24a-24q against drug-
resistant Candida albicans

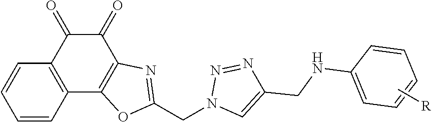

| | | ATCC 90029 | | ATCC 10231 | |
|---|---|---|---|---|---|
| | | MIC | MFC | MIC | MFC |
| Compound | R | (μM) | (μM) | (μM) | (μM) |
| 24a | H | 405.4 | 608.2 | 1621.8 | 2027.2 |
| 24b | 4-F | 516.5 | 581.0 | 1549.4 | 1549.4 |
| 24c | 3-F | 774.7 | 1549.5 | 774.7 | 2582.4 |
| 24d | 2-F | 1537.1 | 2057.7 | 1152.8 | 1801.5 |
| 24e | 4-OMe | 1128.4 | 752.3 | 752.3 | 1504.5 |
| 24f | 3-OMe | 752.3 | 1504.5 | 1003.0 | 1504.5 |
| 24g | 2-OMe | 752.3 | 1504.5 | 752.3 | 2507.6 |
| 24h | 3,5-diOMe | 701.6 | 1169.3 | 701.6 | 1169.3 |
| 24i | 3,4,5- | 657.3 | 1314.5 | 657.3 | 1314.5 |
| | triOMe | | | | |
| 24j | 4-Cl | 744.4 | 2481.2 | 744.7 | 2481.2 |
| 24k | 3-Cl | 1107.6 | 1230.7 | 1107.6 | 1230.7 |
| 241 | 2-Cl | 921.0 | 1730.9 | 921.0 | 1730.9 |
| 24m | 4-Me | 1043.2 | 1564.8 | 1043.2 | 1564.8 |
| 24p | 2-OH | 2067.9 | 1029.8 | 1158.5 | 1810.4 |
| 24q | | 503.6 | 755.4 | 1762.6 | 2014.4 |

| | | | | |
|---|---|---|---|---|
| Fluconazole | 4.0 | 2040.7 | 4081.4 | 4081.4 |

TABLE 9
The MIC and MFC values of Compound 24s-24u against drug-
resistant Candida albicans

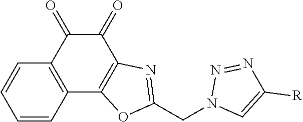

| | | ATCC 90029 | | ATCC 10231 | |
|---|---|---|---|---|---|
| | | MIC | MFC | MIC | MFC |
| Compound | R | (μM) | (μM) | (μM) | (μM) |
| 24s | C₃H₇ | 1282.4 | 2575.1 | 1199.6 | 2254.5 |
| 24t | C₄H₉ | 683.8 | 1536.1 | 1382.5 | 1536.1 |
| 24u | C₅H₁₁ | 732.5 | 2073.9 | 656.4 | 1769.5 |
| Fluconazole | | 4.0 | 2040.7 | 4081.4 | 4081.4 |

### 4. The Inhibitory Activities of Naphtho[2,1-d]Oxazole-4,5-Dione Derivatives Against MRSA

For the inhibitory activities against methicillin-resistant Staphylococcus aureus (MRSA), the inhibition zone was used to test the activities as preliminary screening. Based on the results shown in Table 10, the Compound 23 series had relatively good antibacterial abilities against MRSA. Even at a concentration of 125 μg/ml, the inhibition zone sizes were more than 10 mm. Among them, Compounds 23a and 23b had strong antibacterial abilities. At a concentration of 5000 μg/ml, the sizes of the inhibition zones were 24.70 and 25.20 mm, respectively.

TABLE 10
The inhibition zone sizes (mm) of Compound 23a-23d against
MRSA

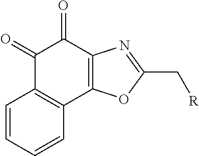

MRSA

| | | 5000 | 2500 | 1250 | 625 | 312 | 125 |
|---|---|---|---|---|---|---|---|
| Compound | R | μg/ml | μg/ml | μg/ml | μg/ml | μg/ml | μg/ml |
| 23a | H | 24.70 | 23.30 | 20.80 | 18.20 | 16.80 | 14.30 |
| 23b | CH₃ | 25.20 | 21.80 | 19.00 | 17.70 | 15.80 | 12.50 |
| 23c | Cl | 19.70 | 18.70 | 16.00 | 13.30 | 11.70 | 10.20 |
| 23d | N₃ | 19.30 | 18.20 | 15.50 | 14.80 | 12.00 | 10.30 |

Table 11 presents the inhibition zone sizes of the Compound 24 series against MRSA. Preliminary results showed that these compounds were able to achieve inhibitory effects on MRSA. Among them, Compounds 24c, 24f, and 24g had the best antibacterial abilities. At a concentration of 5000 μg/ml, the sizes of the inhibition zones reached 16.58, 15.00, and 15.25 mm.

TABLE 11
The ihibition zone sizes (mm) of Compound 24a-24q against MRSA

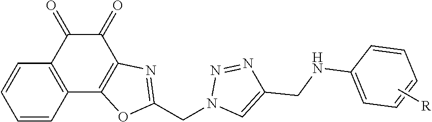

MRSA

| | | 5000 | 2500 | 1250 | 625 | 312 | 125 |
|---|---|---|---|---|---|---|---|
| Compound | R | μg/ml | μg/ml | μg/ml | μg/ml | μg/ml | μg/ml |
| 24a | H | 13.30 | 12.80 | 11.80 | 10.30 | 9.80 | 8.00 |
| 24b | 4-F | 13.50 | 13.30 | 12.30 | 10.00 | 8.80 | 7.50 |
| 24c | 3-F | 16.58 | 15.50 | 14.50 | 12.58 | 11.83 | 10.17 |
| 24e | 4-OMe | 11.70 | 10.00 | 10.30 | 9.30 | 9.00 | 7.30 |
| 24f | 3-OMe | 15.00 | 13.67 | 13.00 | 12.25 | 11.50 | 10.00 |
| 24g | 2-OMe | 15.25 | 14.17 | 13.17 | 11.67 | 10.33 | 8.17 |
| 24h | 3,5- | 14.33 | 13.42 | 12.50 | 12.17 | 11.83 | 10.17 |
| | diOMe | | | | | | |
| 24i | 3,4,5- | 14.33 | 12.83 | 12.17 | 10.17 | 9.17 | 7.67 |
| | triOMe | | | | | | |
| 24j | 4-Cl | 14.08 | 13.67 | 12.67 | 12.33 | 11.83 | 10.00 |
| 24m | 4-Me | 14.75 | 13.67 | 13.50 | 12.42 | 11.83 | 10.67 |
| 24q | | 13.80 | 12.80 | 11.20 | 10.00 | 9.30 | 7.00 |

To remove the effect of physical and chemical properties such as compound solubility and to further evaluate the bactericidal abilities of the synthesized drugs, the present invention tested the minimum inhibitory concentration (MIC) and the minimum bactericidal concentration (MBC) of the two series of compounds against MRSA strains.

Table 12 and Table 13 show the MIC and MBC values of the Compound 23 series and the Compound 24 series against MRSA, and oxacillin, a common antibiotic currently available on the market, was used as a control group. As shown in Table 12, Compounds 23a-23d had significantly improved antibacterial or bactericidal abilities compared to oxacillin sodium. Among them, Compound 23c had the best antibacterial ability, the MIC value was 19.4 μM, which was more than 5 times higher than that of oxacillin sodium. The one with the best bactericidal ability was Compound 23d, and the MBC value was 38.2 μM, which was 16 times better than oxacillin sodium.

As shown in Table 13, the Compound 24 series had obvious antibacterial abilities against MRSA. Among them, Compounds 24j and 24m had the best antibacterial abilities, and the MIC values were 11.0 and 11.6 μM, respectively. Compound 24c had the best antibacterial ability, and the MBC value was 23.8 μM, which was more than 20 times more effective than oxacillin sodium.

TABLE 12
The MIC and MBC values of Compounds 23a-23d against MRSA

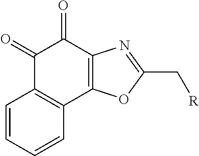

MRSA

| Compound | R | MIC (μM) | MBC (μM) |
|---|---|---|---|
| 23a | H | 30.2 | 365.9 |
| 23b | CH₃ | 28.3 | 42.7 |
| 23c | Cl | 19.4 | 52.4 |
| 23d | N₃ | 38.2 | 38.2 |

| | | |
|---|---|---|
| Oxacillin | 160-630 | 630-1250 |

TABLE 13
The MIC and MBC values of Compounds 24a-24q against MRSA

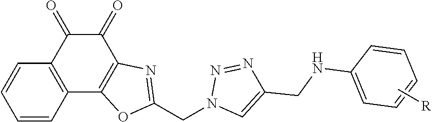

MRSA

| Compound | R | MIC (μM) | MBC (μM) |
|---|---|---|---|
| 24a | H | 59.0 | 126.5 |
| 24b | 4-F | 48.3 | 64.5 |
| 24c | 3-F | 23.8 | 23.8 |
| 24e | 4-OMe | 46.9 | 62.6 |
| 24f | 3-OMe | 29.4 | 35.3 |
| 24g | 2-OMe | 66.7 | 70.6 |
| 24h | 3,5-diOMe | 16.4 | 51.2 |
| 24i | 3,4,5-triOMe | 47.9 | 137.1 |
| 24j | 4-Cl | 11.0 | 139.7 |
| 24m | 4-Me | 11.6 | 36.7 |
| 24q | | 62.8 | 94.3 |

| | | |
|---|---|---|
| Oxacillin sodium | 160-630 | 630-1250 |

The therapeutic effect of compound 24 series on MRSA infection in animal models

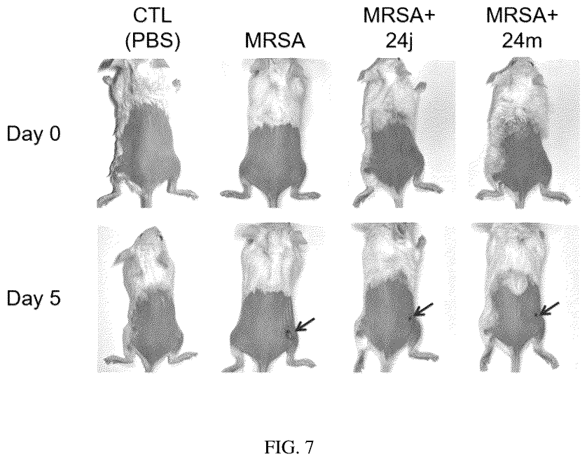

According to the results shown in Table 13, the antibacterial effects of Compounds 24j and 24m were better, so these two compounds were selected for animal experiments. The results of the animal experiment are shown in FIG. 7. The arrow indicates the location of the wound caused by MRSA subcutaneous local infection. After being treated with a local subcutaneous injection of 0.15 mg of Compound 24j or 24m for 5 days, the wound area was analyzed by using Image J imaging software. The image analysis results showed that the wound areas of mice treated with Compound 24j or 24m were reduced by 72.7% and 68.6%, respectively.

The present invention is appropriately described so that it may be practiced with elements or limitations not specifically disclosed herein. The terms that are used to describe are not limiting. No distinction is made between the expressions and descriptions using these terms and any equivalents thereto, but it should be recognized that the rights within the present invention may be modified. Therefore, although the present invention is described in terms of embodiments and other aspects, the contents disclosed herein may be modified and varied by those skilled in the art, and such modifications and variations are deemed to be within the scope of the present invention.

What is claimed is:

1. A compound or a pharmaceutically acceptable salt thereof, wherein the compound has a structure of formula (I): 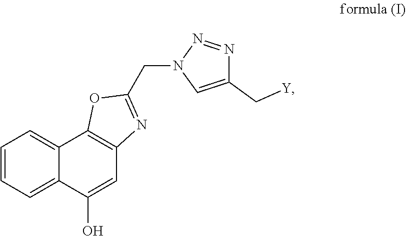
wherein Y is OH, or 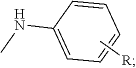
and
R is H, OH, halogen, C₁₋₁₀ alkyl or C₁₋₁₀ alkoxy.

2. The compound or the pharmaceutically acceptable salt thereof of claim 1, wherein the R is H, OH, fluorine or methoxy.

3. An anti-inflammatory method, which comprises administering a composition comprising the compound or the pharmaceutically acceptable salt thereof of claim 1 to a subject suffering from inflammation.

4. A compound or a pharmaceutically acceptable salt thereof, wherein the compound has a structure of formula (II): 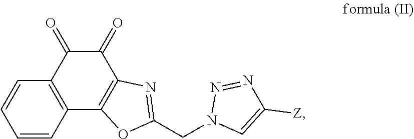
wherein Z is C₁₋₁₀ alkyl, C₁₋₃ alkyl-OH, or 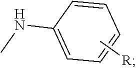
and
R is H, OH, halogen, C₁₋₁₀ alkyl or C₁₋₁₀ alkoxy.

5. The compound or the pharmaceutically acceptable salt thereof of claim 4, wherein the Z is C₃₋₅ alkyl.

6. The compound or the pharmaceutically acceptable salt thereof of claim 4, wherein the Z is C₁ alkyl-OH.

7. The compound or the pharmaceutically acceptable salt thereof of claim 4, wherein the R is H, fluorine, chlorine, methyl or methoxy.

8. A method for treating microbial infections, which comprises administering a composition comprising the compound or the pharmaceutically acceptable salt thereof of claim 4 to a subject suffering from microbial infections, wherein the microorganism comprises Candida or Staphylococcus.

9. The method of claim 8, wherein the Candida comprises drug-resistant and non-drug-resistant Candida albicans, Candida parapsilosis, Candida tropicalis, Candida glabrata, Candida guilliermondii, or Candida krusei.

10. The method of claim 8, wherein the Staphylococcus comprises Staphylococcus aureus, Staphylococcus haemolyticus, Staphylococcus hominis, Staphylococcus intermedius, Staphylococcus saprophyticus, or Staphylococcus saccharolyticus.

11. The method of claim 10, wherein the Staphylococcus aureus comprises methicillin-resistant Staphylococcus aureus, glycopeptide intermediate-sensitive Staphylococcus aureus, vancomycin-resistant Staphylococcus aureus, or vancomycin intermediate-resistant Staphylococcus aureus.
